(19) United States
(12) Patent Application Publication
(10) Pub. No.: US 2022/0127218 A1
(43) Pub. Date: Apr. 28, 2022

(54) METHOD FOR PRODUCING HYDROXYCARBOXYLIC ACID ESTER

(71) Applicants: OSAKA UNIVERSITY, Suita-shi, Osaka (JP); DAICEL CORPORATION, Osaka-shi, Osaka (JP)

(72) Inventors: Tomoo MIZUGAKI, Suita-shi (JP); Kiyotomi KANEDA, Suita-shi (JP); Yasuteru KAJIKAWA, Tokyo (JP); Yuuichirou HIRAI, Tokyo (JP)

(73) Assignees: OSAKA UNIVERSITY, Suita-shi, Osaka (JP); DAICEL CORPORATION, Osaka-shi, Osaka (JP)

(21) Appl. No.: 17/428,127

(22) PCT Filed: Mar. 5, 2019
(86) PCT No.: PCT/JP2019/008686

§ 371 Date: Aug. 3, 2021

(30) Foreign Application Priority Data
Feb. 15, 2019 (JP) 2019-025398

(51) Int. Cl. C07C 67/317; B01J 23/652

(52) U.S. Cl. CPC C07C 67/317; B01J 27/1806; B01J 23/6525

(57) ABSTRACT

An object of the present invention is to provide a method for selectively producing a hydroxycarboxylic acid ester, the method including reducing a dicarboxylic acid monoester by means of a heterogeneous reaction. According to a method for producing a hydroxycarboxylic acid ester in an embodiment of the present invention, a hydroxycarboxylic acid ester represented by Formula (2) is produced by reducing a substrate dicarboxylic acid monoester represented by Formula (1) in the presence of a catalyst.

The catalyst comprises:
metal species including M₁ and M₂; and
a support supporting the metal species, and
wherein
M₁ is rhodium, platinum, ruthenium, iridium or palladium;
M₂ is tin, vanadium, molybdenum, tungsten or rhenium; and
the support is hydroxyapatite, fluorapatite, or hydrotalcite.

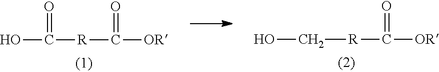

### TECHNICAL FIELD

The present invention relates to a method for producing a hydroxycarboxylic acid ester by reducing a dicarboxylic acid monoester. The present application claims priority from the Japanese Patent Application JP 2019-025398 filed in Japan on Feb. 15, 2019, the contents of which are incorporated herein.

### BACKGROUND ART

There is a strong need for the development of efficient conversion reactions for producing useful chemical products from biomass resources in order to reduce global carbon dioxide emissions. Examples of compounds derived from biomass include dicarboxylic acid monoesters such as monomethyl succinate. Furthermore, hydroxycarboxylic acid esters produced by hydrogenating dicarboxylic acid monoesters are useful as raw materials for plastics, pharmaceutical intermediates, raw materials for cosmetics, and the like. In addition, such hydroxycarboxylic acid esters are also useful as raw materials for fatty acid hydroxycarboxylic ester salts which are useful as emulsifiers.

Known methods for producing a hydroxycarboxylic acid ester by hydrogenating a dicarboxylic acid monoester include one by a homogeneous reaction using borane tetrahydrofuran (BH₃.THF) complex as a reducing agent. However, this method requires an equivalent amount of reducing agent, strict temperature management, and a multi-step reaction, making it unsuitable as a method for industrially producing hydroxycarboxylic acid esters.

Meanwhile, known methods of heterogeneous reaction include one in which a dicarboxylic acid monoester is hydrogenated using a catalyst having Ru and Ge supported on activated carbon (Patent Document 1). However, in this method, the ester group is preferentially reduced; therefore, hydrogenating an adipate, for example, results in mainly 1,6-hexanediol or oxycaproic acid.

That is, a method for selectively producing a hydroxycarboxylic acid ester by reducing a dicarboxylic acid monoester by means of a heterogeneous reaction has yet to be found.

### CITATION LIST

### Patent Document

Patent Document 1: JP 2004-35464 A

### SUMMARY OF INVENTION

### Technical Problem

Therefore, an object of the present invention is to provide a method for selectively producing a hydroxycarboxylic acid ester by reducing a dicarboxylic acid monoester by means of a heterogeneous reaction.

Another object of the present invention is to provide a method for selectively producing a hydroxycarboxylic acid ester by reducing a dicarboxylic acid monoester under mild conditions and with high efficiency.

Yet another object of the present invention is to provide a method for selectively producing a hydroxycarboxylic acid ester by reducing a dicarboxylic acid monoester efficiently using water, which is safe to the human body and environmentally friendly, as a solvent.

Further another object of the present invention is to provide a catalyst used in applications to selectively produce a hydroxycarboxylic acid ester by efficiently reducing a dicarboxylic acid monoester.

### Solution to Problem

As a result of diligent research to solve the problems described above, the inventors of the present invention discovered that using the specific catalyst described below allows the rapid reduction of a dicarboxylic acid monoester to selectively produce a hydroxycarboxylic acid ester. The present invention was completed based on these findings.

That is, the present invention provides a method for producing a hydroxycarboxylic acid ester, the method including reducing a substrate dicarboxylic acid monoester represented by Formula (1) to produce a hydroxycarboxylic acid ester represented by Formula (2) in the presence of a catalyst,

the catalyst comprises:
metal species including M₁ and M₂; and
a support supporting the metal species, and

wherein

M₁ is rhodium, platinum, ruthenium, iridium or palladium;

M₂ is tin, vanadium, molybdenum, tungsten or rhenium; and

the support is hydroxyapatite, fluorapatite, or hydrotalcite.

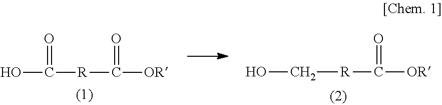

where R represents a single-bond or divalent hydrocarbon group while R′ represents a monovalent hydrocarbon group.

The present invention also provides the method for producing a hydroxycarboxylic acid ester, wherein a selectivity of the hydroxycarboxylic acid ester represented by Formula (2) below in the total amount of the reaction products is not less than 70% while a selectivity of a lactone represented by Formula (3) below in the total amount of the reaction products is not greater than 5% at the time when a conversion ratio of the substrate reaches not less than 90%,

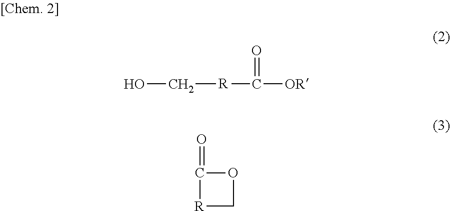

where R represents a single-bond or divalent hydrocarbon group while R′ represents a monovalent hydrocarbon group.

The present invention also provides the method for producing a hydroxycarboxylic acid ester, wherein the catalyst contains M₁ and M₂ as metal species in a ratio from 0.05 to 1 mol of M₂ per 1 mol of M₁.

The present invention also provides the method for producing a hydroxycarboxylic acid ester, wherein the amount of the catalyst (in terms of the M₁ metal) is from 0.01 to 30 mol % of the substrate.

The present invention also provides the method for producing a hydroxycarboxylic acid ester, wherein a reduction reaction is carried out in the presence of water.

The present invention also provides a catalyst comprising;
metal species including M₁ and M₂; and
a support supporting the metal species, and

wherein

M₁ is rhodium, platinum, ruthenium, iridium or palladium;

M₂ is tin, vanadium, molybdenum, tungsten or rhenium; and

the support is hydroxyapatite, fluorapatite, or hydrotalcite,

wherein the catalyst being used to reduce a dicarboxylic acid monoester to form a corresponding hydroxycarboxylic acid ester.

### Advantageous Effects of Invention

With the production method according to an embodiment of the present invention, a hydroxycarboxylic acid ester can be efficiently and selectively produced from a dicarboxylic acid monoester under mild conditions and in a one-step manner.

In addition, with the production method according to an embodiment of the present invention, it is possible to efficiently and selectively produce a hydroxycarboxylic acid ester using water, which is safe to the human body and environmentally friendly, as a solvent.

Furthermore, hydroxycarboxylic acid esters formed in this manner are useful as raw materials for plastics, pharmaceutical intermediates, raw materials for cosmetics, and the like. In addition, hydroxycarboxylic acid esters formed in this manner are also useful as raw materials for fatty acid hydroxycarboxylic ester salts which are useful as emulsifiers. Therefore, the production method according to an embodiment of the present invention is suitable as a method for producing hydroxycarboxylic acid ester industrially.

Furthermore, the catalyst according to an embodiment of the present invention, wherein the catalyst which includes M₁ and M₂ supported on hydroxyapatite, fluorapatite, or hydrotalcite, can be suitably used as a catalyst for reducing a dicarboxylic acid monoester to selectively produce a hydroxycarboxylic acid ester using water, which is safe to the human body and environmentally friendly, as a solvent.

### DESCRIPTION OF EMBODIMENTS

### Catalyst

According to the method for producing a hydroxycarboxylic acid ester in an embodiment of the present invention, at least one catalyst is used, the catalyst including M₁ and M₂ described below, which serve as metal species, supported on a support described below.

(M₁): rhodium, platinum, ruthenium, iridium or palladium

(M₂): tin, vanadium, molybdenum, tungsten or rhenium

(Support): hydroxyapatite, fluorapatite, or hydrotalcite

M₁ and M₂ that are supported on a support may be a simple metal, or may be a metal salt, a metal oxide, a metal hydroxide, a metal complex, or the like.

The amount of M₁ supported (in terms of metal) is, for example, approximately from 1 to 50 wt. %, preferably from 1 to 20 wt. %, and particularly preferably from 1 to 10 wt. %, of the support. When the catalyst supports M₁ in an excess amount, the catalytic activity reaches saturation and levels off, and does not achieve the effect of promoting the reaction further. Meanwhile, when the catalyst supports M₁ in an amount less than the range described above, the catalyst may not readily exhibit sufficient catalytic activity.

The amount of M₂ supported (in terms of metal) is, for example, approximately from 0.01 to 20 wt. %, preferably from 0.01 to 10 wt. %, particularly preferably from 0.01 to 1 wt. %, most preferably from 0.05 to 0.8 wt. %, especially preferably from 0.1 to 0.6 wt. %, of the support. When the amount of M₂ supported is out of the range described above, it tends to be difficult to selectively produce a hydroxycarboxylic acid ester.

The catalyst in an embodiment of the present invention is considered to have an active site at the interface of M₁ and M₂. Furthermore, when either one of M₁ and M₂ is in excess, catalytic activity may decrease while the yield of hydroxycarboxylic acid ester tends to decline; this may be because the metal species in excess covers the other metal species, reducing the interface and shrinking the active site.

Therefore, the amounts of M₁ and M₂ supported are preferably in a specific range, and the amount of M₂ supported per 1 mol of M₁ is preferably, for example, from 0.05 to 1 mol. Furthermore, the upper limit of the amount of M₂ supported per 1 mol of M₁ is preferably 0.5 mol, more preferably 0.4 mol, most preferably 0.35 mol, and especially preferably 0.3 mol. The lower limit of the amount of M₂ supported per 1 mol of M₁ is preferably 0.07 mol, more preferably 0.1 mol, most preferably 0.15 mol, and especially preferably 0.2 mol.

In the catalyst according to an embodiment of the present invention, the amount of metal species other than M₁ and M₂ is, for example, not greater than 200 mol %, preferably not greater than 150 mol %, more preferably not greater than 100 mol %, further preferably not greater than 70 mol %, even further preferably not greater than 50 mol %, even further more preferably not greater than 30 mol %, particularly preferably not greater than 10 mol %, most preferably not greater than 5 mol %, and especially preferably not greater than 1 mol %. When the amount of metal species other than M₁ and M₂ exceeds the range described above, the effect of the present invention may not be readily achieved; this may be because the shrunken active site.

In an embodiment of the present invention, M₁ and M₂ are used while being supported on a support. Having M₁ and M₂ supported on a support can increase the interface area of M₁ and M₂, and thereby increasing exposure of the active site.

Furthermore, in an embodiment of the present invention, since a catalyst formed by having M₁ and M₂ supported on a support is used, the catalyst can be easily separated from the reaction products by physical separation methods such as filtration or centrifugation after completion of the reaction; the catalyst separated from the reaction products and recovered can be reused as it is, or after, for example, being washed or dried. In an embodiment of the present invention, since an expensive catalyst can be used repeatedly as described above, the cost of producing a hydroxycarboxylic acid ester can be greatly reduced.

The support is preferably hydroxyapatite or fluorapatite, particularly preferably hydroxyapatite, from the perspective that a hydroxycarboxylic acid ester can be selectively produced from a dicarboxylic acid monoester at a high yield.

The support is preferably hydroxyapatite or hydrotalcite, particularly preferably hydroxyapatite, from the perspective that a hydroxycarboxylic acid ester can be selectively produced from a dicarboxylic acid monoester at a high yield.

For the hydroxyapatite, commercially available products such as the product with the trade name “Tricalcium Phosphate” (available from Wako Pure Chemical Industries, Ltd.) can be suitably used.

The catalyst according to an embodiment of the present invention can be suitably used as a reduction catalyst for reducing a dicarboxylic acid monoester to produce a hydroxycarboxylic acid ester.

Method for Preparing Catalyst

The catalyst in an embodiment of the present invention can be prepared, for example, by an impregnation method.

An impregnating method is a method for supporting a metal species on a support, including immersing a support in a solution (for example, an aqueous solution) prepared by dissolving a compound containing the metal species mentioned above (i.e. a metal compound) in a solvent (for example, water), impregnating the support with the metal compound, and then subjecting to calcination. The supported amount of the metal species can be controlled by adjusting, for example, the concentration of the metal compound in the solution, or the immersion time of the support.

The catalyst in an embodiment of the present invention can be prepared by: a sequential impregnation method, in which a support is sequentially impregnated with a solution prepared by dissolving a compound containing M₁ in a solvent (hereinafter, it may be referred to as “M₁-containing solution”) and a solution prepared by dissolving a compound containing M₂ in a solvent (hereinafter, it may be referred to as “M₂-containing solution”); or, a co-impregnation method in which a support is simultaneously impregnated with an M₁-containing solution and an M₂-containing solution. When preparing the catalyst using a co-impregnation method, calcination may be performed after impregnating the support in a mixed solution of an M₁-containing solution and an M₂-containing solution; on the other hand, when preparing the catalyst using a sequential impregnation method, it is preferable to perform calcination each time after immersing the support in an M₁-containing solution and an M₂-containing solution one after another.

Among these, in an embodiment of the present invention, a catalyst formed by supporting M₁ and M₂ on a support by a co-impregnation method is particularly preferable from the perspective that a hydroxycarboxylic acid ester can be produced selectively.

For example, a catalyst in which Pt as M₁ and Mo as M₂ are supported on hydroxyapatite as the support by a co-impregnation method (for example, Pt—Mo/HAP) can be prepared by: immersing hydroxyapatite in a solution which is formed by dissolving a Pt compound (such as H₂PtCl₆) and an Mo compound [such as (NH₄)₆Mo₇O₂₄.4H₂O] in water; then, distilling off the solvent and calcining the hydroxyapatite.

The temperature at which the support is immersed in the solution is, for example, approximately from 10 to 80° C.

The time of immersing the support in the solution is, for example, approximately from 1 to 30 hours, preferably from 1 to 5 hours.

Calcination is carried out by, for example, performing heating at from 300 to 700° C. for from 1 to 5 hours using a muffle furnace or the like.

Furthermore, reduction treatment may be further performed after calcination. Examples of reducing agents used for the reduction treatment include hydrogen (H₂).

The temperature and time of the reduction treatment are, for example, approximately from 0.5 to 5 hours (preferably from 0.5 to 2 hours) at a temperature of from 0 to 600° C. (preferably from 100 to 200° C.).

The catalyst prepared by the preparation method described above may then be subjected to, for example, a washing treatment (washing with water, an organic solvent, or the like), or a drying treatment (drying by vacuum drying, or the like).

### Substrate

The dicarboxylic acid monoester represented by Formula (1) below is used as the substrate in an embodiment of the present invention.

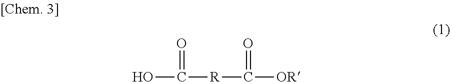

In the formula, R represents a single-bond or divalent hydrocarbon group while R′ represents a monovalent hydrocarbon group.

The divalent hydrocarbon group in R includes a divalent aliphatic hydrocarbon group, a divalent alicyclic hydrocarbon group, a divalent aromatic hydrocarbon group, and a divalent group formed by two or more groups selected from the aforementioned groups bonded together.

Examples of the divalent aliphatic hydrocarbon group include: straight-chain or branched alkylene groups having from 1 to 10 carbons (preferably from 1 to 6 carbons), such as a methylene group, a methylmethylene group, a dimethylmethylene group, an ethylene group, a propylene group, and a trimethylene group; and straight-chain or branched alkenylene groups having from 2 to 10 carbons (preferably from 2 to 6 carbons), such as a vinylene group, a 1-methylvinylene group, a propenylene group, a 1-butenylene group, a 2-butenylene group, a 1-pentenylene group, and a 2-pentenylene group.

Examples of the divalent alicyclic hydrocarbon group include:

cycloalkylene groups having from 3 to 10 carbons (preferably from 3 to 6 carbons), such as a cyclopentylene group, a cyclohexylene group (such as a 1,2-cyclohexylene group, a 1,3-cyclohexylene group, and a 1,4-cyclohexylene group), and a cycloheptylene group; and cycloalkenylene groups having from 3 to 10 carbons (preferably from 3 to 6 carbons), such as a cyclopropenylene group, a cyclobutenylene group, a cyclopentenylene group, a cyclohexenylene group, and a cyclooctenylene group.

Examples of the divalent aromatic hydrocarbon group include: arylene groups having from 6 to 20 carbons, such as a phenylene group (for example, an o-phenylene group, an m-phenylene group, and a p-phenylene group), a biphenylene group, a naphthylene group, a binaphthylene group, and an anthracenylene group.

Examples of the divalent group formed by two or more groups selected from the aforementioned hydrocarbon groups bonded together include: cyclohexylenebis(methylene) [for example, 1,2-cyclohexylenebis(methylene), 1,3-cyclohexylenebis(methylene), and 1,4-cyclohexylenebis(methylene)]; and phenylenebis(methylene) [for example, 1,2-phenylenebis(methylene), 1,3-phenylenebis(methylene), and 1,4-phenylenebis(methylene)].

The monovalent hydrocarbon group in R′ includes a monovalent aliphatic hydrocarbon group, a monovalent alicyclic hydrocarbon group, a monovalent aromatic hydrocarbon group, and a monovalent group formed by two or more groups selected from the aforementioned groups bonded together.

Examples of the monovalent aliphatic hydrocarbon group include: straight-chain or branched alkyl groups having from 1 to 10 carbons (preferably from 1 to 5 carbons), such as a methyl group, an ethyl group, a propyl group, an isopropyl group, a butyl group, a pentyl group, an octyl group, a 2-ethylhexyl group, and a decyl group; and straight-chain or branched alkenyl groups having from 2 to 10 carbons (preferably from 2 to 5 carbons), such as a vinyl group, an allyl group, a propenyl group, a 1-butenyl group, a 2-butenyl group, a 1-pentenyl group, and a 2-pentenyl group.

Examples of the monovalent alicyclic hydrocarbon group include: cycloalkyl groups having from 3 to 10 carbons (preferably from 3 to 6 carbons), such as a cyclopentyl group, a cyclohexyl group, and a cycloheptyl group; cycloalkenyl groups having from 3 to 10 carbons (preferably from 3 to 6 carbons), such as a cyclopropenyl group, a cyclobutenyl group, a cyclopentenyl group, a cyclohexenyl group, and a cyclooctenyl group.

Examples of the monovalent aromatic hydrocarbon group include aryl groups having from 6 to 20 carbons, such as a phenyl group, a biphenyl group, a naphthyl group, a binaphthyl group, and an anthracenyl group.

Examples of the monovalent group formed by two or more groups selected from the aforementioned hydrocarbon groups bonded together include a cyclohexylmethyl group and a benzyl group.

The aforementioned monovalent hydrocarbon group and the divalent hydrocarbon group may have one or two or more substituents. Examples of the substituent include a C₁₋₅ alkoxy group, a C₆₋₁₀ aryloxy group, a C₇₋₁₁ aralkyloxy group, an oxo group, a halogen atom, and a halo C₁₋₅ alkyl group.

### Method for Producing Hydroxycarboxylic Acid Ester

In the method for producing a hydroxycarboxylic acid ester according to an embodiment of the present invention, the carboxyl group of the substrate, which is a dicarboxylic acid monoester, is reduced (preferably reduced with molecular hydrogen) in the presence of the catalyst described above to produce the corresponding hydroxycarboxylic acid ester.

The catalyst selectively reduces only carboxyl groups even if ester groups are present in the substrate, and the hydroxycarboxylic acid ester represented by Formula (2) below can be selectively formed from the dicarboxylic acid monoester represented by Formula (1) below [1].

In addition, since the reduction reaction of the carboxyl group in the dicarboxylic acid monoester represented by Formula (1) below proceeds faster than the intramolecular dehydration condensation reaction, the generation of the lactone represented by Formula (3) below as a byproduct can be suppressed [2]

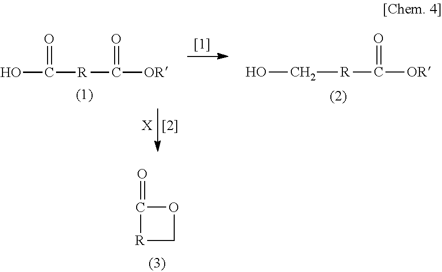

(where R and R′ are the same as described above)

The amount of the catalyst (in terms of the metal M₁ contained in the catalyst) is, for example, approximately from 0.01 to 30 mol %, preferably from 0.1 to 10 mol %, particularly preferably from 0.5 to 5 mol %, most preferably from 1 to 5 mol %, of the substrate.

Furthermore, the amount of the catalyst (in terms of the metal M₂ contained in the catalyst) is, for example, approximately from 0.01 to 10 mol %, preferably from 0.05 to 5 mol %, and particularly preferably from 0.1 to 2 mol %, of the substrate.

When the catalyst is used in the range described above, the substrate can be efficiently hydrogenated under mild conditions to selectively produce a hydroxycarboxylic acid ester. When the amount of the catalyst is less than the range described above, the yield of hydroxycarboxylic acid ester tends to decrease.

Hydrogen used for the reduction reaction (or hydrogenation reaction) can be supplied by, for example, a method including carrying out the reaction in a hydrogen atmosphere, or a method including bubbling hydrogen gas.

In an embodiment of the present invention, since the catalyst mentioned above is used, reduction of the carboxyl group contained in the substrate can proceed rapidly under mild conditions, and the hydrogen pressure during the reduction reaction is, for example, not greater than 10 MPa, preferably not greater than 8 MPa, more preferably not greater than 6 MPa, and particularly preferably not greater than 5 MPa (such as from 1 to 5 MPa).

The reaction temperature of the reduction reaction is, for example, from 50 to 200° C., preferably from 100 to 180° C., particularly preferably from 120 to 160° C., and most preferably from 120 to 150° C.

The reaction time of the reduction reaction is, for example, approximately from 1 to 36 hours, preferably from 5 to 30 hours, and particularly preferably from 5 to 20 hours.

The reduction reaction can be performed by any method, such as a batch method, a semi-batch method, and a continuous method.

The reduction reaction is preferably carried out in the liquid phase. In other words, the reduction reaction according to an embodiment of the present invention is preferably a liquid-phase reaction. This is because dicarboxylic acid monoester has a high boiling point, and when the reduction reaction is performed in the gas phase, reaction products tend to decompose and the yield of hydroxycarboxylic acid ester tends to decrease.

When the reaction is carried out in the liquid phase, examples of solvent include: water; alcohol-based solvents such as methanol, ethanol, 2-propanol, and 1-butanol; ether-based solvents such as 1,4-dioxane, THF, 1,2-dimethoxyethane, and diethyl ether; hydrocarbon solvents such as toluene, hexane, and dodecane; halogenated hydrocarbon solvents such as 1,2-dichloroethane and dichloromethane. One of these solvents can be used alone or two or more in combination.

Among these solvents, in an embodiment of the present invention, water is preferable from the perspective that it is safe to the human body and environmentally friendly. That is, the reduction reaction in an embodiment of the present invention is preferably performed in the presence of water. In addition, in an embodiment of the present invention, even if the lactone represented by Formula (3) is produced as a byproduct through the reaction of [2] described above, when the reduction reaction is carried out in the presence of water, the ester ring-opening reaction of the byproduct lactone proceeds, producing the hydroxycarboxylic acid ester represented by Formula (2). Therefore, in an embodiment of the present invention, it is especially preferable that reduction reaction is carried out in the presence of water from the perspective that the hydroxycarboxylic acid ester represented by Formula (2) can be formed at a high yield.

The amount of water used in the total amount of the solvent is, for example, not less than 1 wt. %, more preferably not less than 5 wt. %, more preferably not less than 10 wt. %, even more preferably not less than 30 wt. %, further more preferably not less than 50 wt. %, particularly preferably not less than 70 wt. %, most preferably not less than 80 wt. %, and especially preferably not less than 90 wt. %. Therefore, the amount of solvent used besides water (for example, an organic solvent, particularly an ether-based solvent such as THF) in the total amount of the solvent is, for example, preferably not greater than 90 wt. %, more preferably not greater than 80 wt. %, even more preferably not greater than 70 wt. %, further preferably not greater than 50 wt. %, even further preferably not greater than 30 wt. %, particularly preferably not greater than 20 wt. %, more particularly preferably not greater than 10 wt. %, most preferably not greater than 5 wt. %, and especially preferably not greater than 1 wt. %.

The amount of solvent used is preferably in a range such that the initial concentration of the substrate is, for example, approximately from 0.01 to 10 wt. % when reacted using a batch method.

In an embodiment of the present invention, since the catalyst described above is used, reduction reaction of the substrate can proceed rapidly even if one or two or more selected from the followings does not exist in the reaction system: acids such as hydrochloric acid, sulfuric acid, phosphoric acid, and acetic acid, and bases such as sodium hydroxide, potassium hydroxide, sodium carbonate, sodium hydrogen carbonate, potassium carbonate, and potassium hydrogen carbonate. Also, in an embodiment of the present invention, although an acid or base mentioned above may be used, the amount used (the total amount when two or more are used) is preferably, for example, less than 0.001 mol per 1 mol of the substrate, and it is particularly preferable that none of the acids or bases is actually used. This is because when the acid or base mentioned above is present in the reaction system beyond the range described above, the post-treatment needs to include neutralization treatment, which produces salt as a by-product, and removing the by-produced salt causes loss of the product. Furthermore, the corrosiveness of the acids or bases mentioned above limits the material of the reactor to be used.

After the completion of the reaction, the resulting reaction products can be separated and purified by: a separation method, such as filtration, concentration, distillation, extraction, crystallization, recrystallization, and column chromatography; or a separation method in combination thereof.

The method for producing a hydroxycarboxylic acid ester according to an embodiment of the present invention facilitates efficient conversion of the substrate even under mild conditions. The conversion ratio of the substrate after 30 hours (preferably after 20 hours) from the start of the reaction is, for example, not less than 80%, preferably not less than 90%, and particularly preferably not less than 95%.

Furthermore, when the catalyst described above is used, the reduction reaction of the carboxyl group proceeds extremely quickly; thus, generation of lactone as a byproduct due to the proceeding of intramolecular dehydration condensation reaction of the dicarboxylic acid monoester can be prevented or suppressed.

Therefore, when the conversion ratio of the dicarboxylic acid monoester, which is the substrate, reaches at least 90%, the selectivity of the hydroxycarboxylic acid ester represented by Formula (2) above in the total amount of reaction products is, for example, not less than 70%, preferably not less than 80%, particularly preferably not less than 85%, and most preferably not less than 90%. Meanwhile, the selectivity of the lactone represented by Formula (3) above is, for example, not greater than 5%, preferably not greater than 3%, and particularly preferably not greater than 1%.

Therefore, with the method for producing a hydroxycarboxylic acid ester according to an embodiment of the present invention, a hydroxycarboxylic acid ester can be selectively produced at a high yield in a simple one-step method while suppressing the generation of lactone as a byproduct and using water, which is safe to the human body and environmentally friendly, as a solvent.

### EXAMPLES

Hereinafter, the present invention will be described more specifically with reference to examples, but the present invention is not limited by these examples.

### Example 1

### Preparation of Catalyst: Co-Impregnation Method

0.0898 g of H₂PtCl₆ and 0.0088 g of (NH₄)₆Mo₇O₂₄.4H₂O were dissolved in 50 mL of water to prepare a solution; 1 g of hydroxyapatite (HAP, trade name “Tricalcium Phosphate”, available from Wako Pure Chemical Industries, Ltd.) was immersed in the resulting solution for 4 hours under room temperature (25° C.). After immersion, water was distilled off in a rotary evaporator under reduced pressure to prepare a powder. The formed powder was then calcined in an air atmosphere in a muffle furnace at 500° C. for 3 hours to prepare Catalyst (1) [Pt—Mo/HAP, amount of Pt supported: 4 wt. %, amount of Mo supported: 0.485 wt. %, Mo/Pt (molar ratio)=0.25].

### Examples 2 to 5

1 mmol of the substrate as described in the table below, 100 mg of Catalyst (1) [Pt that is 2 mol % of the substrate, Mo that is 0.5 mol % of the substrate (in terms of metal)], and 3 mL of water were charged in an autoclave having a Teflon (trade name) inner cylinder and reacted at 110° C. for a number of hours as described in the table below under the condition of hydrogen pressure of 5 MPa to produce reaction products. The conversion ratio (conv. [%]) of the substrate was measured using HPLC, and the yield of each one of the reaction products was measured using a gas chromatograph mass spectrometer (GC-MS).

The results are summarized and shown in the table below.

TABLE 1

| | | time | conversion | product yield |
|---|---|---|---|---|
| entry | substrate | (h) | (%) | (%) |
| 2 | 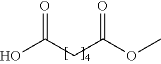 | 18 | >99 | 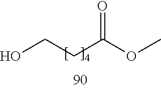 |
| | | | | 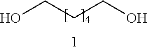 |
| | | | | 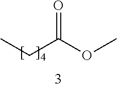 |
| 3 | 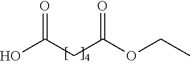 | 14 | >99 | 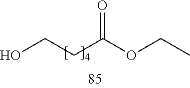 |
| | | | | 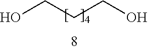 |
| | | | | 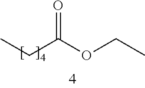 |
| 4 | 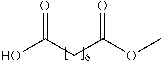 | 12 | >99 | 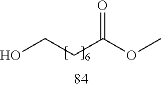 |
| | | | | 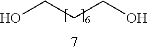 |
| | | | | 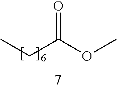 |
| | | | | 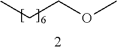 |
| 5 | 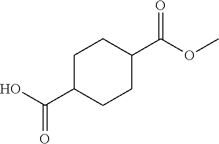 | 18 | 96 | 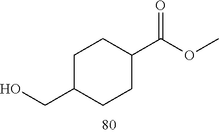 |
| | | | | 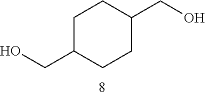 |
| | | | | 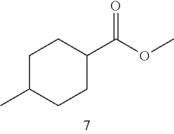 |

To summarize the above, configurations and variations according to an embodiment of the present invention will be described below.

[1] A method for producing a hydroxycarboxylic acid ester, the method including reducing a substrate dicarboxylic acid monoester represented by Formula (1) to produce a hydroxycarboxylic acid ester represented by Formula (2) in the presence of a catalyst,

the catalyst comprises:
metal species including M₁ and M₂; and
a support supporting the metal species, and

wherein

M₁ is rhodium, platinum, ruthenium, iridium or palladium;

M₂ is tin, vanadium, molybdenum, tungsten or rhenium; and

the support is hydroxyapatite, fluorapatite, or hydrotalcite.

[2] The method for producing a hydroxycarboxylic acid ester according to [1], wherein an amount of M₁ supported (in terms of metal) is from 1 to 50 wt. % of the support.

[3] The method for producing a hydroxycarboxylic acid ester according to [1] or [2], wherein an amount of M₂ supported (in terms of metal) is from 0.01 to 20 wt. % of the support.

[4] The method for producing a hydroxycarboxylic acid ester according to any one of [1] to [3], wherein an amount of M₂ supported per 1 mol of M₁ is from 0.05 to 1 mol.

[5] The method for producing a hydroxycarboxylic acid ester according to any one of 111 to [4], wherein an amount of a metal species other than M₁ and M₂ is not greater than 200 mol % of the total supported amount of M₁ and M₂.

[6] The method for producing a hydroxycarboxylic acid ester according to any one of [1] to [5], wherein the support is hydroxyapatite or fluorapatite.

[7] The method for producing a hydroxycarboxylic acid ester according to any one of [1] to [5], wherein the support is hydroxyapatite or hydrotalcite.

[8] The method for producing a hydroxycarboxylic acid ester according to any one of [1] to [7], wherein M₁ is rhodium, platinum, ruthenium, or iridium.

[9] The method for producing a hydroxycarboxylic acid ester according to any one of 111 to [7], wherein M₁ is platinum, ruthenium, iridium, or palladium.

[10] The method for producing a hydroxycarboxylic acid ester according to any one of [1] to [7], wherein M₁ is rhodium, platinum, or ruthenium.

[11] The method for producing a hydroxycarboxylic acid ester according to any one of [1] to [7], wherein M₁ is platinum, ruthenium, or iridium.

[12] The method for producing a hydroxycarboxylic acid ester according to any one of [1] to [7], wherein M₁ is rhodium or platinum.

[13] The method for producing a hydroxycarboxylic acid ester according to any one of [1] to [7], wherein M₁ is platinum or ruthenium.

[14] The method for producing a hydroxycarboxylic acid ester according to any one of [1] to [7], wherein M₁ is platinum or iridium.

[15] The method for producing a hydroxycarboxylic acid ester according to any one of [1] to [7], wherein M₁ is platinum or palladium.

[16] The method for producing a hydroxycarboxylic acid ester according to any one of [1] to [15], wherein M₂ is vanadium, molybdenum, tungsten, or rhenium.

[17] The method for producing a hydroxycarboxylic acid ester according to any one of 111 to [15], wherein M₂ is molybdenum, tungsten, or rhenium.

[18] The method for producing a hydroxycarboxylic acid ester according to any one of 111 to [15], wherein M₂ is vanadium, molybdenum, or tungsten.

[19] The method for producing a hydroxycarboxylic acid ester according to any one of [1] to [15], wherein M₂ is molybdenum or tungsten.

[20] The method for producing a hydroxycarboxylic acid ester according to any one of [1] to [15], wherein M₂ is molybdenum or rhenium.

[21] The method for producing a hydroxycarboxylic acid ester according to any one of [1] to [15], wherein M₂ is vanadium or molybdenum.

[22] The method for producing a hydroxycarboxylic acid ester according to any one of [1] to [21], wherein a conversion ratio of the substrate after 30 hours from a start of the reaction is not less than 80%.

[23] The method for producing a hydroxycarboxylic acid ester according to any one of [1] to [22], wherein a selectivity of the hydroxycarboxylic acid ester represented by Formula (2) in the total amount of the reaction products is not less than 70% while a selectivity of a lactone represented by Formula (3) in the total amount of the reaction products is not greater than 5% at the time when a conversion ratio of the substrate reaches not less than 90%.

[24] The method for producing a hydroxycarboxylic acid ester according to any one of [1] to [23], wherein the catalyst contains M₁ and M₂ as metal species in a range of from 0.05 to 1 mol of M₂ per 1 mol of M₁.

[25] The method for producing a hydroxycarboxylic acid ester according to any one of [1] to [24], wherein an amount of the catalyst (in terms of the metal M₁) is from 0.01 to 30 mol % of the substrate.

[26] The method for producing a hydroxycarboxylic acid ester according to any one of [1] to [25], wherein a reduction reaction is performed in the presence of water.

[27] The method for producing a hydroxycarboxylic acid ester according to [26], wherein a proportion of water in the total amount of solvent is not less than 70 wt. %.

[28] The method for producing a hydroxycarboxylic acid ester according to any one of [1] to [27], wherein an amount of acid and base used (the total amount when two or more types are contained) is less than 0.001 mol per 1 mol of the substrate.

[29] The method for producing a hydroxycarboxylic acid ester according to [28], wherein the acid is at least one acid selected from the group consisting of hydrochloric acid, sulfuric acid, phosphoric acid, and acetic acid.

[30] The method for producing a hydroxycarboxylic acid ester according to [28] or [29], wherein the base is at least one base selected from the group consisting of sodium hydroxide, potassium hydroxide, sodium carbonate, sodium hydrogen carbonate, potassium carbonate, and potassium hydrogen carbonate.

[31] A catalyst comprising;
metal species including M₁ and M₂; and
a support supporting the metal species, and

wherein

M₁ is rhodium, platinum, ruthenium, iridium or palladium;

M₂ is tin, vanadium, molybdenum, tungsten or rhenium; and

the support is hydroxyapatite, fluorapatite, or hydrotalcite,

wherein the catalyst being used to reduce a dicarboxylic acid monoester to form a corresponding hydroxycarboxylic acid ester.

[32] The reduction catalyst for dicarboxylic acid monoesters according to [31], wherein an amount of M₁ supported in terms of metal is from 1 to 50 wt. % of the support.

[33] The reduction catalyst for dicarboxylic acid monoesters according to [31] or [32], wherein an amount of M₂ supported in terms of metal is from 0.01 to 20 wt. % of the support.

[34] The reduction catalyst for dicarboxylic acid monoesters according to any one of [31] to [33], wherein an amount of M₂ supported per 1 mol of M₁ is from 0.05 to 1 mol.

[35] The reduction catalyst for dicarboxylic acid monoesters according to any one of [31] to [34], wherein an amount of a metal species other than M₁ and M₂ is not greater than 200 mol % of the total amount of M₁ and M₂.

[36] The reduction catalyst for dicarboxylic acid monoesters according to any one of [31] to [35], wherein the support is hydroxyapatite or fluorapatite.

[37] The reduction catalyst for dicarboxylic acid monoesters according to any one of [31] to [35], wherein the support is hydroxyapatite or hydrotalcite.

[38] The reduction catalyst for dicarboxylic acid monoesters according to any one of [31] to [37], where M₁ is rhodium, platinum, ruthenium, or iridium.

[39] The reduction catalyst for dicarboxylic acid monoesters according to any one of [31] to [37], wherein M₁ is platinum, ruthenium, iridium, or palladium.

[40] The reduction catalyst for dicarboxylic acid monoesters according to any one of [31] to [37], where M₁ is rhodium, platinum, or ruthenium.

[41] The reduction catalyst for dicarboxylic acid monoesters according to any one of [31] to [37], wherein M₁ is platinum, ruthenium, or iridium.

[42] The reduction catalyst for dicarboxylic acid monoesters according to any one of [31] to [37], wherein M₁ is rhodium or platinum.

[43] The reduction catalyst for dicarboxylic acid monoesters according to any one of [31] to [37], wherein M₁ is platinum or ruthenium.

[44] The reduction catalyst for dicarboxylic acid monoesters according to any one of [31] to [37], wherein M₁ is platinum or iridium.

[45] The reduction catalyst for dicarboxylic acid monoesters according to any one of [31] to [37], wherein M₁ is platinum or palladium.

[46] The reduction catalyst for dicarboxylic acid monoesters according to any one of [31] to [45], wherein M₂ is vanadium, molybdenum, tungsten, or rhenium.

[47] The reduction catalyst for dicarboxylic acid monoesters according to any one of [31] to [45], wherein M₂ is molybdenum, tungsten, or rhenium.

[48] The reduction catalyst for dicarboxylic acid monoesters according to any one of [31] to [45], wherein M₂ is vanadium, molybdenum, or tungsten.

[49] The reduction catalyst for dicarboxylic acid monoesters according to any one of [31] to [45], wherein M₂ is molybdenum or tungsten.

[50] The reduction catalyst for dicarboxylic acid monoesters according to any one of [31] to [45], wherein M₂ is molybdenum or rhenium.

[51] The reduction catalyst for dicarboxylic acid monoesters according to any one of [31] to [45], wherein M₂ is vanadium or molybdenum.

### INDUSTRIAL APPLICABILITY

According to the production method in an embodiment of the present invention, a hydroxycarboxylic acid ester can be efficiently and selectively produced from a dicarboxylic acid monoester under mild conditions in a one-step manner using water, which is environmentally friendly, as a solvent.

Furthermore, hydroxycarboxylic acid esters formed in this manner are useful as raw materials for plastics, pharmaceutical intermediates, raw materials for cosmetics, and the like.

1. A method for producing a hydroxycarboxylic acid ester, the method including reducing a substrate dicarboxylic acid monoester represented by Formula (1) to produce a hydroxycarboxylic acid ester represented by Formula (2) in the presence of a catalyst, 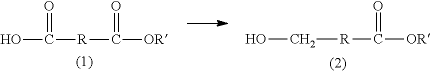
where R represents a single-bond or divalent hydrocarbon group while R′ represents a monovalent hydrocarbon group; wherein
the catalyst comprises:
metal species including M₁ and M₂; and
a support supporting the metal species, and
wherein
M₁ is rhodium, platinum, ruthenium, iridium or palladium;
M₂ is tin, vanadium, molybdenum, tungsten or rhenium; and
the support is hydroxyapatite, fluorapatite, or hydrotalcite.

2. The method for producing a hydroxycarboxylic acid ester according to claim 1, wherein a selectivity of the hydroxycarboxylic acid ester represented by Formula (2) in the total amount of the reaction products is not less than 70% while a selectivity of a lactone represented by Formula (3) in the total amount of the reaction products is not greater than 5% at the time when a conversion ratio of the substrate reaches not less than 90%, 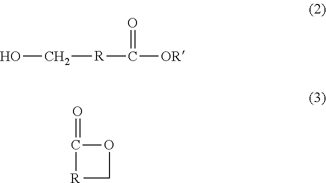
where R represents a single-bond or divalent hydrocarbon group while R′ represents a monovalent hydrocarbon group.

3. The method for producing a hydroxycarboxylic acid ester according to claim 1, wherein the catalyst contains M₁ and M₂ as metal species in a range of from 0.05 to 1 mol of M₂ per 1 mol of M₁.

4. The method for producing a hydroxycarboxylic acid ester according to claim 1, wherein an amount of the catalyst in terms of the metal M₁ is from 0.01 to 30 mol % of the substrate.

5. The method for producing a hydroxycarboxylic acid ester according to claim 1, wherein a reduction reaction is performed in the presence of water.

6. A catalyst comprising;
metal species including M₁ and M₂; and
a support supporting the metal species, and
wherein
M₁ is rhodium, platinum, ruthenium, iridium or palladium;
M₂ is tin, vanadium, molybdenum, tungsten or rhenium; and
the support is hydroxyapatite, fluorapatite, or hydrotalcite,
wherein the catalyst being used to reduce a dicarboxylic acid monoester to form a corresponding hydroxycarboxylic acid ester.

7. The method for producing a hydroxycarboxylic acid ester according to claim 1, wherein an amount of M₁ in terms of metal is from 1 to 50 wt. % of the support.

8. The method for producing a hydroxycarboxylic acid ester according to claim 1, wherein an amount of M₂ in terms of metal is from 0.01 to 20 wt. % of the support.

9. The method for producing a hydroxycarboxylic acid ester according to claim 1, wherein an amount of a metal species other than M₁ and M₂ is not greater than 30 mol % of the total amount of M₁ and M₂.

10. The method for producing a hydroxycarboxylic acid ester according to claim 1, wherein M₁ is rhodium, platinum, or ruthenium.

11. The method for producing a hydroxycarboxylic acid ester according to claim 1, wherein M₂ is vanadium, molybdenum, tungsten, or rhenium.

12. The method for producing a hydroxycarboxylic acid ester according to claim 1, wherein a conversion ratio of the substrate after 30 hours from a start of the reaction is not less than 80%.

13. The method for producing a hydroxycarboxylic acid ester according to claim 1, wherein a proportion of water in the total amount of solvent is not less than 70 wt. %.

14. The method for producing a hydroxycarboxylic acid ester according to claim 1, wherein acid and/or base may be used as a catalyst, but the total amount of acid and base used is less than 0.001 mol per 1 mol of the substrate.

15. The method for producing a hydroxycarboxylic acid ester according to claim 14, wherein the acid is at least one acid selected from the group consisting of hydrochloric acid, sulfuric acid, phosphoric acid, and acetic acid.

16. The method for producing a hydroxycarboxylic acid ester according to claim 14, wherein the base is at least one base selected from the group consisting of sodium hydroxide, potassium hydroxide, sodium carbonate, sodium hydrogen carbonate, potassium carbonate, and potassium hydrogen carbonate.
